(19) United States
(12) Patent Application Publication
(10) Pub. No.: US 2023/0414404 A1
(43) Pub. Date: Dec. 28, 2023

(54) HEATING PLATE WITH RECESS

(71) Applicant: Kamedi GmbH, Karlsruhe (DE)

(72) Inventors: Armin MEYER, Karlsruhe (DE); Christof REUTER, Karlsruhe (DE); Lukas LIEDTKE, Karlsruhe (DE); Stefan HOTZ, Karlsruhe (DE)

(73) Assignee: Kamedi GmbH, Karlsruhe (DE)

(21) Appl. No.: 18/212,525

(22) Filed: Jun. 21, 2023

(30) Foreign Application Priority Data
Jun. 28, 2022 (DE) 10 2022 116 008.4

(51) Int. Cl. A61F 7/00

(52) U.S. Cl. CPC A61F 7/007; A61F 2007/0052

(57) ABSTRACT

A heating plate for the thermal-medical treatment of skin includes an acting section for being applied to the skin on a top side of the heating plate; a connecting section for attaching the heating plate to a printed circuit board on a bottom side of the heating plate; and a recess on the bottom side of the heating plate. A device for the thermal-medical treatment of skin and a method of manufacturing a heating plate are also disclosed.

The present invention relates to a heating plate for the thermal-medical treatment of skin. The present invention further relates to a device for the thermal-medical treatment of skin as well as a method for manufacturing a heating plate.

Skin irritations as can be induced, for example, by insect bites, plants, fish, seaweed, jellyfish or other kinds of cnidarians, can be cured and/or their effects alleviated by a local heat treatment. As soon as possible after the occurrence of the skin irritation or the triggering event, the affected skin area is heated, for example to a temperature between 45° C. and 65° C. The temperature is maintained for a few seconds. The heating stimulates blood circulation and suppresses a skin reaction that can lead to the skin irritation. Pain and itching are relieved.

Due to the fact that the above-mentioned skin irritations often occur in the outdoor area, portable devices for carrying out a corresponding heat treatment have become available in the meantime. For example, WO 2019/020144 A1 discloses a couplable portable device for the thermal-medical treatment of skin. The device comprises a treatment attachment that can be coupled via an interface to a mobile data processing unit, preferably a smartphone or a tablet. In the coupled state, a housing of the treatment attachment overlapping the treatment attachment is provided with a heating element directly underneath the surface facing away from the data processing unit. The heating element is coupled to a temperature sensor. The heating element, with the interposition of a control unit, is connected to an electrically conductive interface or, via a Bluetooth connection, to the data-processing unit in such a way that the heating element is energized via one or more accumulators integrated into the data processing unit so that the heating element can be heated to a temperature predefined via the control unit.

In such devices, regardless of whether they are designed to be coupled to a mobile device or as stand-alone devices with their own control electronics and power supply, so-called heating plates are usually used to transmit heat from a heating element to the skin area to be treated. To heat the heating plate, it is thermally contacted with a heating element. For example, a heating resistor can be used whose heat is directed into the heating plate. For this purpose, the heating plate in particular can be soldered onto a printed circuit board, whereby the heat generated by the heating element is directed into the heating plate via the soldering points. Both the heating element and the heating plate are advantageously arranged in immediate vicinity and are minimally dimensioned so that as much of the heat generated as possible can be applied to the skin surface to be treated. Due to the metallic soldering connection between the various components, the thermal resistance is low.

Based on the foregoing, according to prior art the design is usually such that a heating plate is soldered onto one side of a printed circuit board and a heating element is attached to the other side of the printed circuit board. Thermal contact is then achieved via metallized holes in the area of the contact points of the heating element and the heating plate. Heat can be directed into the heating plate through the metallized holes. Advantageously, these holes are additionally filled with tin or another suitable metal to further reduce thermal resistance.

The heating plate itself is usually made of ceramic, with a metallic material, in particular copper, being applied to one side of the heating plate for contacting purposes. In other words, the heating plate comprises a layer or section for contacting the skin on one side and a section for attaching to a printed circuit board on the other side. In the manufacturing process, this contacting layer or section is often applied to a larger ceramic plate and baked. Only after completion of this connection process is the larger plate then cut into individual small heating plates.

Disadvantages of previous heating plates for this area of application are the high manufacturing costs and the low flexibility of the design when using the heating plate within the device for thermal-medical treatment. In addition, the production is often comparatively complex and cost-intensive. On the other hand, due to the manufacturing processes used and the specifications for the application, only a few degrees of freedom remain with regard to the attachment and processing of the heating plate in the device for thermal-medical treatment of skin.

Based on the foregoing, the problem faced by the present invention is to provide a heating plate that can be efficiently manufactured and allows for flexibility with regard to the attachment in the device for thermal-medical treatment of skin. In particular, a heating plate is to be provided which allows for a simplified and flexible assembly process.

To solve this problem, in a first aspect, the present invention relates to a heating plate for the thermal-medical treatment of skin, comprising:
an acting section for being applied to the skin on a top side of the heating plate;
a connecting section for attaching the heating plate to a printed circuit board on a bottom side of the heating plate; and
a recess on the bottom side of the heating plate.

In another aspect, the present invention relates to a device for the thermal-medical treatment of skin comprising:
a printed circuit board, in particular a flexible printed circuit board;
a heating plate according to any one of the preceding claims, attached to the printed circuit board; and
a heating element thermally connected to the heating plate for heating the heating plate to a treatment temperature.

In a further aspect, the present invention relates to a method of manufacturing a heating plate as previously described, comprising a step of creating the recess, a further recess and/or an edge section by ablative laser processing of the heating plate.

In a further aspect, the present invention relates to a method of manufacturing a heating plate, in particular a heating plate as previously described, comprising a step of creating the recess, a further recess and/or an edge section by pressing a ceramic powder into a mold.

Preferred embodiments of the invention are described in the dependent claims. It is understood that the above features and those to be explained below can be used not only in the combination indicated in each case, but also in other combinations or on a stand-alone basis, without departing from the scope of the above invention. In particular, the heating plate, the device as well as the methods can be designed according to the embodiments described for the heating plate, the device and the methods in the dependent claims.

According to the invention, the heating plate is to be provided with a recess on its bottom side. A recess is understood to mean in particular an area of missing material. In particular, the recess creates a cavity or free space between the printed circuit board and the heating plate when the heating plate is attached to the printed circuit board. The connecting section designates the section or layer of the heating plate through which the thermal and mechanical connection with the printed circuit board is established. The acting section is the area of the heating plate that transfers the heat to the skin surface of the person to be treated. The heat generated by the heating element is transferred to the acting section via the connecting section. The acting section can then transfer the heat to the skin of the person to be treated. The cavity or free space created by the recess may be located within the connecting section and/or within the acting section of the heating plate. The top side of the heating plate is applied to the skin. The bottom side of the heating plate is connected to a printed circuit board. A heating plate or a plate is understood to mean in particular a shape or a structure which has a significantly smaller extension in one direction than in the other two directions and is thus flat.

According to the invention, the heating plate and the recess are to be produced by ablative laser processing or by pressing a ceramic powder into a mold. Both ablative laser processing and pressing of a ceramic powder into a mold can enable an efficient production of the recess. Compared to previous approaches, in particular the mechanical generation of a recess, the costs are reduced and the efficiency of the production is significantly improved.

Compared to previous heating plates, which are solid and have no recess on their bottom side, the recess in the heating plate according to the invention allows further degrees of freedom with regard to the attachment to the printed circuit board. For example, light can be introduced into the heating plate through the recess, so that a luminous heating plate is possible. By means of a luminous heating plate, the application can be improved for the user, since the user can identify and treat the skin area to be treated even in the dark. In contrast to the arrangement of a luminous ring around the heating plate, this results in cost and application advantages. In addition, this results in an improved and more comprehensive illumination of the skin area in question with reduced energy consumption. At the same time, the design freedom of the rest of the device can be improved by a luminous heating plate, since no translucent plastic elements have to be installed.

In addition, the recess of the heating plate according to the invention allows for the use of a one-sided assembly process. Since a heating element or other components can be arranged in the recess, for example, one-sided assembly is sufficient, which allows cost advantages to be realized. In previous approaches, two-sided assembly is often required, with the heating element being arranged on the side of the printed circuit board opposite the heating plate.

The manufacturing processes of ablative laser processing or pressing of a ceramic powder according to the invention enable the production of more complex shapes, so that the degrees of freedom in further processing steps are increased. In addition, good surfaces can be produced that enable an efficient heat transmission to the skin area to be treated and avoid skin irritation. Furthermore, the achievable smooth surfaces can facilitate assembly. Particularly advantageous is the use of a flex board onto which the heating plate is soldered, so that control and power supply are not situated in the same plane as the heating plate, but can be arranged in an angled position relative to this plane. This allows for further design flexibility.

In a preferred embodiment, the recess is designed for accommodating a component on the printed circuit board when the heating plate is attached to the printed circuit board. In particular, the recess may be designed to accommodate an LED, preferably a soldered surface-mounted LED. Advantageously, the recess has a size that is sufficient to accommodate a component. In other words, the dimensioning of the recess enables a component to be accommodated on the printed circuit board. On the one hand, this component can be the heating element, so that the heating element can be attached on the same side of the printed circuit board as the heating plate due to the recess. This makes it possible to abstain from two-sided assembly. On the other side, it is possible to arrange an LED in the recess. The LED generates light which can then be passed through the heating plate, resulting in a luminous heating plate and illumination of the skin area to be treated. It is understood that the recess may also be dimensioned to accommodate multiple components. For example, a heating element and one or more LEDs can be arranged in the recess. Compared to previous approaches with solid heating plates, this results in reduced manufacturing costs and further degrees of freedom with regard to structuring and design. Furthermore, the components arranged in the recess are protected from moisture and other environmental influences.

In a preferred embodiment, the recess comprises areas with different depths. In other words, there are at least two areas in the recess which have different depths perpendicular to the plane of the printed circuit board or perpendicular to the heating plate. Different depths allow the recess or the dimension of the recess to be adapted to one or more components arranged within the recess. For example, due to the use of several depths, the recess can be dimensioned in such a way that two components can be accommodated in the recess and at the same time the heating plate is not unnecessarily weakened and thus retains its mechanical stability and good thermal conductivity. It is also possible for a shape of a component to be mimicked by means of different depths of the recess, so that the dimensioning of the recess can be as small as possible in order to enable a minimum dimensioning of the heating plate itself with a sufficient amount of material of the heating plate and sufficient heat absorption capacity.

In a preferred embodiment, the recess extends through the connecting section and into the acting section. In principle, it is possible for the recess to be provided only within the connecting section. Advantageously, however, the design or depth is chosen such that it extends through the connecting section into the acting section. This allows a component to be accommodated in the recess and/or light to be transmitted through the recess and the acting section.

In a preferred embodiment, the recess in an area within the acting section is open in exactly one spatial direction. In other words, an area of the recess in the acting section corresponds to a cutout that is open on one side. Additionally or alternatively, the recess is designed to be closed off from an environment by the printed circuit board. Additionally or alternatively, the recess has, at least in a partial area, a bone-shaped cross-sectional area with a centrally arranged central area and two exterior areas widened relative to the central area. A recess which is closed off from an environment enables a cavity to be created under the heating plate when the latter is attached to a printed circuit board. In other words, the recess is not designed as a cutout at the edge of the heating plate, but is closed on all sides either by material of the heating plate or by the printed circuit board itself when attached. The use of a bone-shaped cross-sectional area, in particular on the bottom side of the heating plate, enables components to be accommodated with as much as possible amount of material of the heating plate. Preferably, the recess has a bone-shaped cross-sectional area in an area of the recess within the acting section. The cross-sectional area is to be understood as an area parallel to the heating plate or parallel to the printed circuit board in the area where the heating plate is attached to the printed circuit board. At least in a partial area, the recess has a bone-shaped cross-sectional area or bone-shaped cross-section, which enables adaptation to a shape of a component. In addition, the stability of the heating plate is improved. By making the heating plate as solid as possible, the dimensioning of the heating plate can be reduced if sufficient heat capacity is given.

In a preferred embodiment, the acting section is made of a ceramic material. Preferably, the connecting section is formed of a different material that is connected to the ceramic material using a firing process. Ceramic is biocompatible, so that heat can be transferred to the skin without causing skin irritation. In addition, a high thermal conductivity is provided. In particular, a ceramic material with good thermal conductivity can be used. For example, aluminum nitride or aluminum oxide can be used. For the attachment of the ceramic to a printed circuit board, the use of a different material is preferably intended in the connecting section, which can be connected to the ceramic material using a baking process. In particular, a metal layer may be provided as a connecting section, preferably a copper layer.

In a preferred embodiment, the acting section in the area of the recess has a thickness of between 0.2 mm and 0.8 mm, preferably about 0.4 mm. Additionally or alternatively, the heating plate has a maximum thickness between mm and 1.5 mm. Further additionally or alternatively, the acting section has rounded edges. The specified thickness of the acting section or the maximum thickness of the heating plate allows for sufficient mechanical stability combined with sufficient heat absorption capacity. Nevertheless, the thickness is small enough to provide for a sufficiently quick heatability along with a comparatively low energy input, as required for a mobile application of the device for thermal-medical treatment of skin according to the invention. By using rounded edges in the acting section, injuries or additional skin irritations due to scratches can be avoided when treating the skin. In addition, this results in improved mechanical stability, since edging and thus higher force application is avoided.

In a preferred embodiment, the heating plate comprises a further recess on the top side of the heating plate within the acting section. The further recess preferably has a depth which is selected such that a bottom of the further recess is brought into contact with the skin when the acting section is applied to the skin by pressing in the skin. In this respect, the further recess is located opposite the first recess on the top side of the heating plate. For example, a symbol in the form of a recess can be provided on the top side of the heating plate. Thereby, it is preferably intended that nevertheless a substantial part of the acting section or of the top side of the heating plate can be brought into contact with the skin in order to further enable an efficient thermal-medical treatment of the skin through use of heat. In this respect, the recess on the top side of the heating plate is of comparatively small depth. This leads to a higher degree of freedom of design.

In a preferred embodiment, the connecting section comprises a metal layer for soldering the heating plate onto the printed circuit board, in particular a copper layer. Preferably, the metal layer comprises several areas not electrically connected to one another for soldering onto multiple soldering surfaces of the printed circuit board. In particular, the connecting section may correspond to a metal layer. The metal layer may be applied to a ceramic layer forming the acting section, for example by baking. Via the metal layer, a mechanical and thermal connection of the heating plate to the printed circuit board can be established. In this respect, the use of a copper layer is particularly advantageous due to its high electrical and thermal conductivity. By using several areas that are not electrically connected to one another, a direct coupling of a heating element, for example a heating resistor, can be achieved. The multiple soldering surfaces of the printed circuit board to which the heating plate is soldered by means of the connecting section are thermally connected to the soldering surfaces of the printed circuit board to which the heating element or the heating resistor is coupled. In this respect, the heat of the heating element is transferred directly to the heating plate.

In a preferred embodiment, the heating plate comprises an edge section which is set back relative to the acting section on the top side of the heating plate. The edge section preferably circulates the acting section step-like. Additionally or alternatively, the edge section is made of the same material as the acting section. The edge section is thus preferably made of the material of the acting section or the material of the connecting section. In this respect, an edge section is understood to be an area in which the heating plate has a smaller thickness or, when attached to the printed circuit board, a smaller height than in a central area. In other words, it can be provided, for example, that a step circulates around the heating plate. This allows for a high degree of stability when attaching the heating plate to the printed circuit board, as well as a reduction of the mechanical susceptibility of the heating plate or the device during use.

In a preferred embodiment, the device comprises a component attached to the printed circuit board in the recess of the heating plate. In particular, the component may be an LED.

In a preferred embodiment of the method according to the invention, parallel processing of multiple heating plates takes place in one panel. Parallel processing leads to cost savings and enables an efficient production.

In this context, a heating plate or a plate is understood to be a plate-like structure which has a larger dimension in one plane than in a height perpendicular to this plane. An acting section or also a connecting section are in particular layers which are connected to one another. A recess corresponds to an area of missing material and may also be referred to as a cavity or cutout. The heating plate has a top side and a bottom side, with the top side obviously denoting the side opposite the bottom side. The thickness between the top and bottom sides is substantially less than the dimension in the other two spatial directions. A printed circuit board is understood in this context to mean in particular a circuit board on which electronic components can be arranged. A printed circuit board can in particular be a flexible board or flex board or flexible printed circuit board. A metal surface or a contacting metal surface or a soldering surface of the printed circuit board is understood to mean a soldering pad or a metallized area for the attachment of electronic components. A surface of a printed circuit board may be both a top side and a bottom side.

The invention is described and explained in more detail below with reference to some selected embodiments in connection with the accompanying drawings. They show:

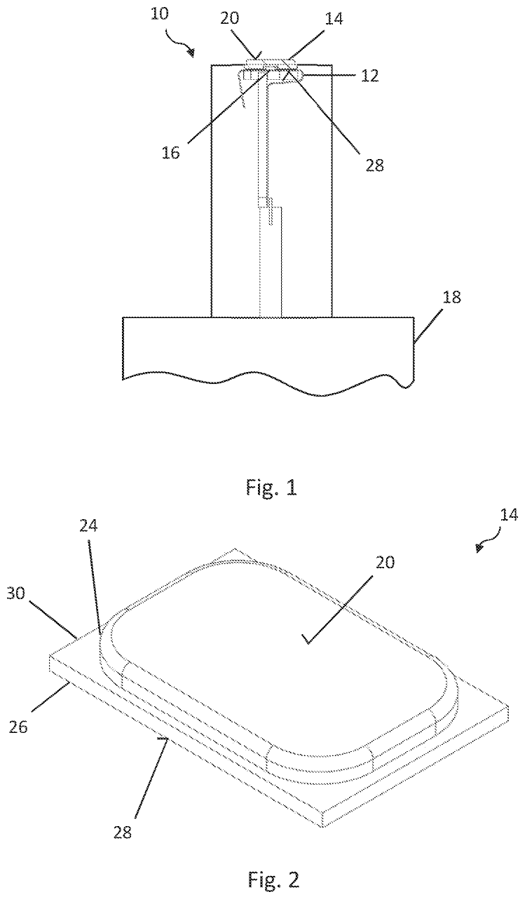

FIG. 1 a schematic view of the design of a device for the thermal-medical treatment of skin according to the invention;

FIG. 2 a schematic perspective view of the top side of a heating plate according to the invention;

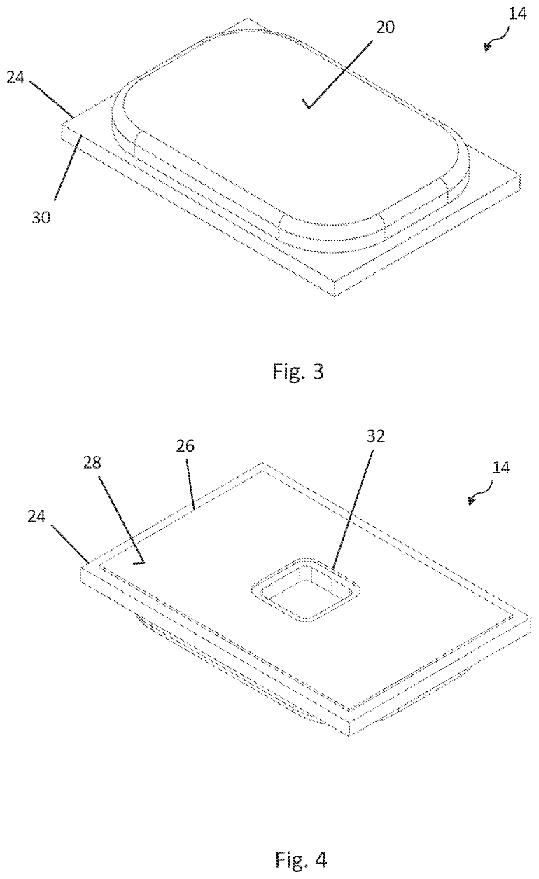

FIG. 3 a schematic perspective view of the top side of a further embodiment of a heating plate according to the invention;

FIG. 4 a schematic perspective view of the bottom side of an embodiment of a heating plate according to the invention with a connecting section and a rectangular recess;

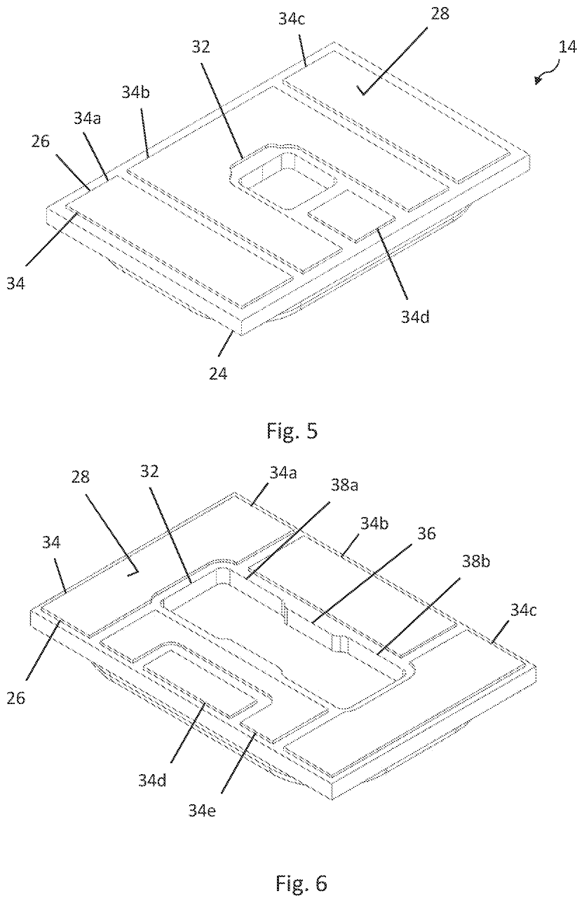

FIG. 5 a schematic view of a bottom side of a heating plate according to the invention with multiple areas of the connecting section that are not electrically connected to one another;

FIG. 6 a schematic view of a recess having a bone-shaped cross-sectional area;

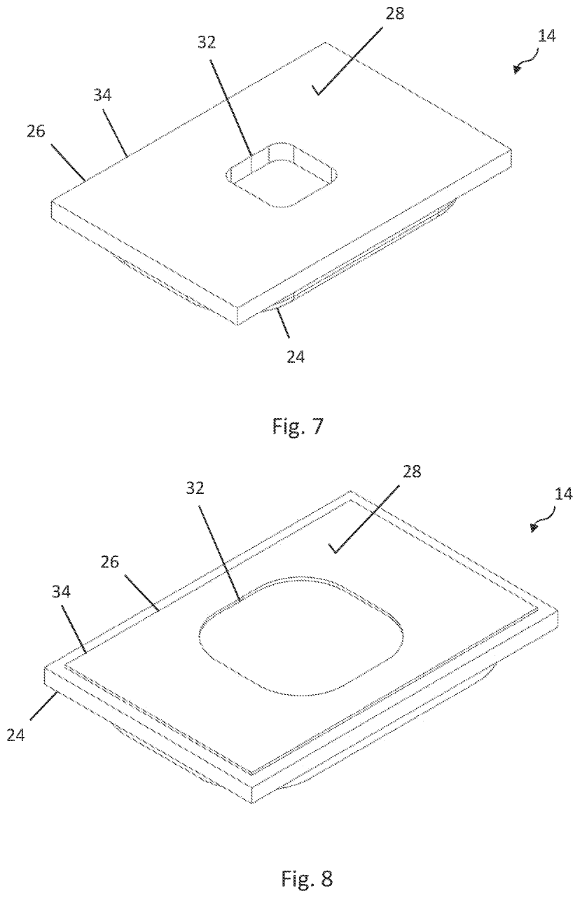

FIG. 7 a schematic view of a recess in the connecting section;

FIG. 8 a schematic view of a recess in the connecting section with small depth;

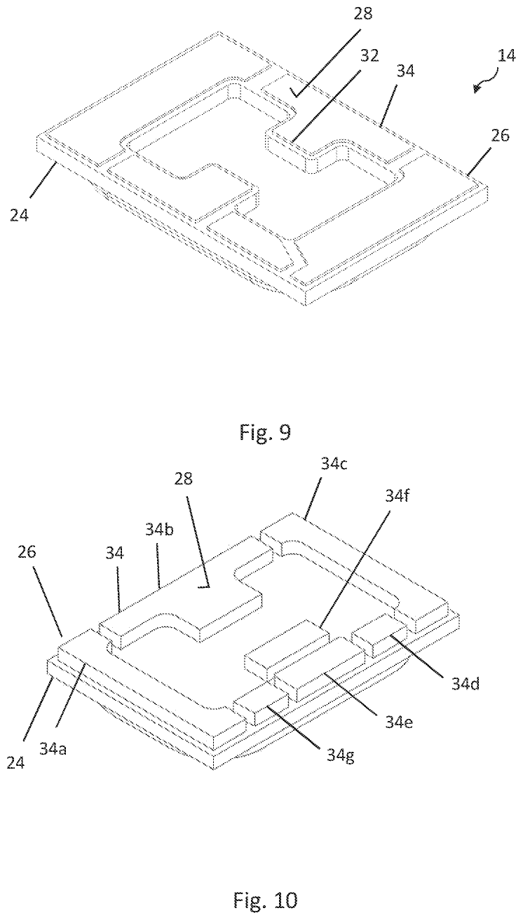

FIG. 9 a schematic view of a bone-shaped recess extending into the connecting section and into the acting section;

FIG. 10 a schematic view of a bone-shaped recess extending only into the connecting section;

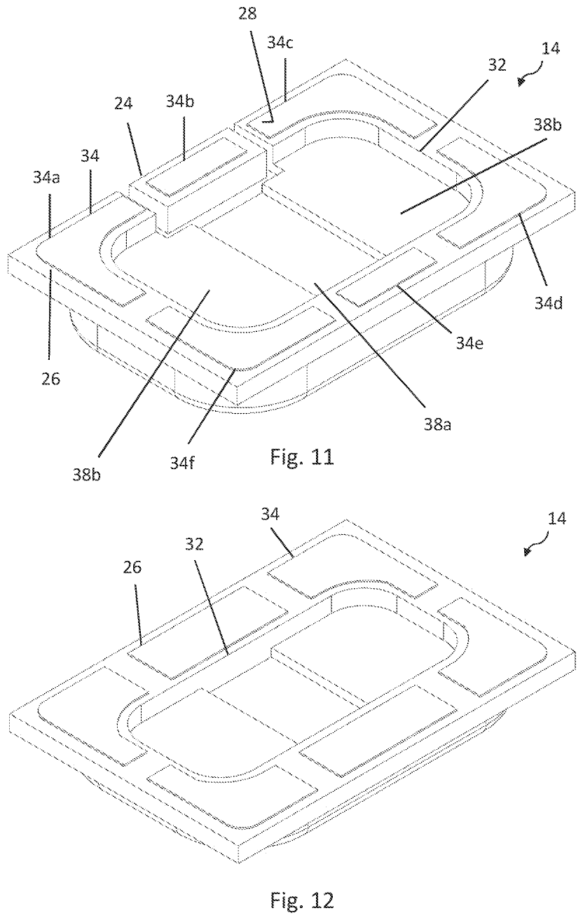

FIG. 11 a schematic view of a recess with areas of varying depth;

FIG. 12 a schematic view of another embodiment with areas of the recess having different depths;

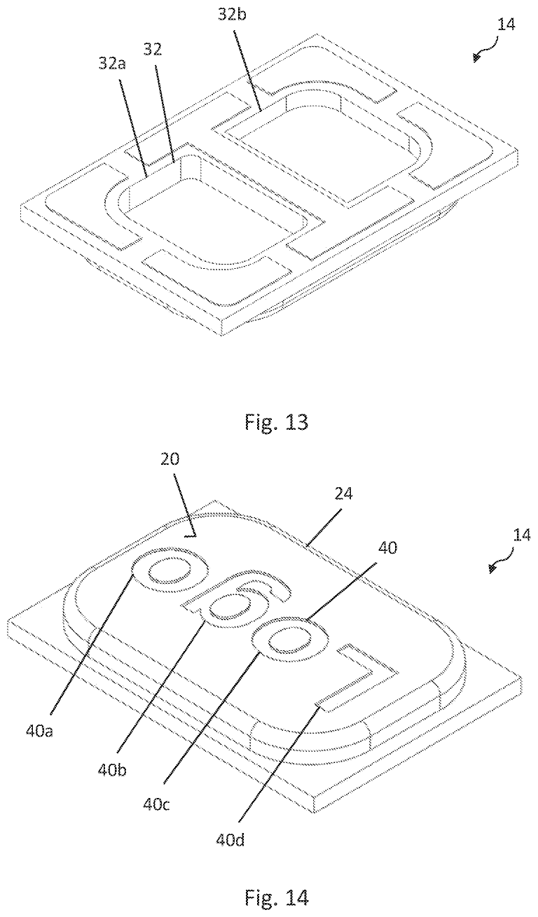

FIG. 13 a schematic view of an embodiment with a recess divided in two parts;

FIG. 14 a schematic view of a top side of a heating plate according to the invention with a further recess;

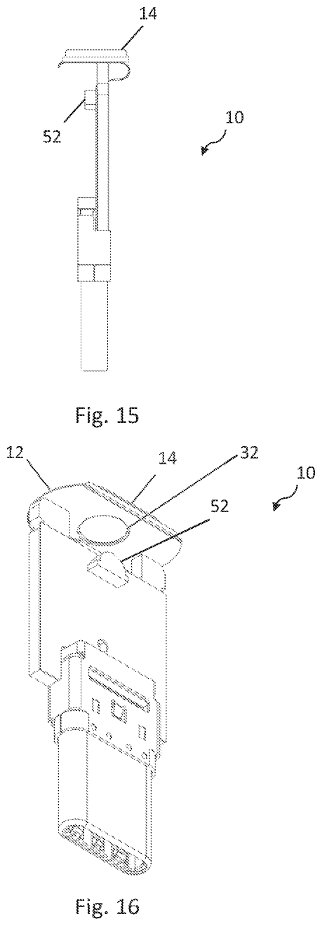

FIG. 15 a schematic view of an arrangement of the heating plate in a device according to the invention;

FIG. 16 a perspective view of a further arrangement in a device according to the invention; and

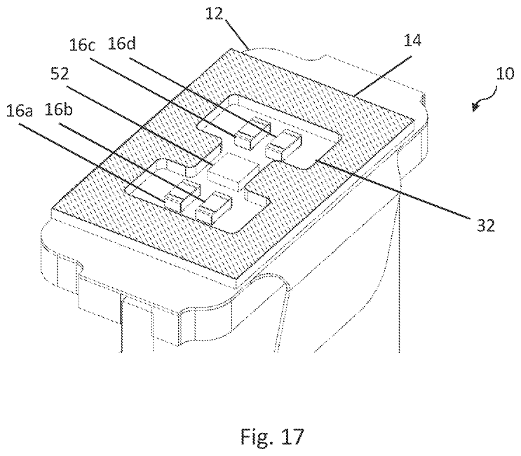

FIG. 17 a perspective sectional view of a further arrangement in a device according to the invention.

FIG. 1 shows a schematic view of a device 10 according to the invention for the thermal-medical treatment of skin. The illustration is to be understood as a sectional view in which the components of the device 10 are visualized. The device 10 comprises a printed circuit board 12, a heating plate 14, and a heating element 16. In the illustrated embodiment, the device 10 is designed as a couplable portable device for coupling to a mobile device 18, in particular a smartphone or a tablet. For example, the device 10 may be configured to plug into a USB-C or lightning port of the mobile device 18 to be powered and/or controlled from the mobile device 18. It is understood that other embodiments of the device 10 according to the invention are also possible, in particular a stand-alone device.

In the illustrated embodiment, the heating plate 14 and the heating element 16 are attached on different sides of the printed circuit board 12, which is designed as a flexible printed circuit board. In this case, the heating plate 14 serves to contact the skin (not shown) and to transfer heat to the skin. The heating element 16 can be designed, for example, as a heating resistor or a heating transistor and serves for heat generation. The heat is directed through the printed circuit board 12 into the heating element 16. On its top side 20 the heating plate 14 comprises an acting section to be placed on the skin. On a bottom side 28, the heating plate 14 comprises a connecting section for attaching the heating plate 14 to the printed circuit board 12, for example by soldering.

In FIG. 2, a heating plate 14 according to the invention is shown schematically in perspective view. In particular, the top side 20 of the heating plate 14 can be seen. The acting section 24 arranged on the top side 20 of the heating plate 14 is designed to apply the heating plate 14 to the skin. In particular, the acting section 24 may be formed of a ceramic material. For example, aluminum nitride or aluminum oxide can be used. The acting section 24 may have rounded edges to allow for an easy application without risk of injury. Furthermore, the connection-compliant heating plate 14 comprises a connecting section 26 by means of which the heating plate 14 can be fixed to a printed circuit board. The connecting section 26 is arranged on the bottom side 28 of the heating plate 14. The heating plate 14 can have a total thickness of between 0.5 mm and 1.5 mm, for example.

In the embodiment shown in FIG. 2, the heating plate 14 has an edge section 30 which is arranged on the top side 20 of the heating plate 14 and is set back relative to the acting section 24. In the illustrated embodiment, the edge section 30 is designed as a circumferential step, which facilitates assembly.

FIG. 3 shows a schematic view of an embodiment of a heating plate 14 according to the invention, in which the acting section 24 on the top side 20 comprises both the offset upper area with the rounded edge and the edge section 30. Thereby, the edge section 30 is formed of the same material as the acting section. Not shown in FIG. 3 is the connecting section, which is not visible in the illustration due to the perspective view.

In comparison of FIGS. 2 and 3, the circumferential edge section 30 in FIG. 2 is thus part of the connecting section 26, whereas the circumferential edge section 30 in FIG. 3 is part of the acting section 24.

FIG. 4 shows a schematic view of the bottom side 28 of a heating plate 14 according to the invention. In the embodiment shown in FIG. 4, the acting section 24 corresponds to a 3D-formed ceramic layer. The connecting section 26 corresponds to a metal layer connected to this ceramic layer, in particular a copper layer. A recess 32 is provided on the bottom side 28 of the heating plate 14 according to the invention. In the illustrated embodiment, the recess 32 extends through the connecting section 26 into the acting section 24. In various embodiments of the invention, the acting section 24 may have a thickness of between 0.2 mm and 1 mm in the area of the recess 32, for example, preferably approximately 0.4 mm. This ensures sufficient stability.

The invention provides that the heating plate 14 is manufactured by ablative laser processing and/or by pressing a ceramic powder into a mold. By means of these manufacturing processes, it is possible for the heating plate 14 to obtain a 3D shaping. In particular, by applying such manufacturing processes to a ceramic material, a variety of different shapes can be realized. By using a 3D manufacturing process, such as ablative laser processing, a recess 32 can be created.

The recess 32 can accommodate, for example, a component that is attached to the printed circuit board. The heating plate 14 is arranged in the manner of a lid, so to speak, above a component on the printed circuit board. The component is then located in the recess 32. For example, the recess 32 can thereby be dimensioned such that an LED can be arranged therein. In this case, it is possible for light to be generated by this LED, with, for example, a ceramic material being translucent, so that, due to the generation of light in the recess 32, illumination of the heating plate 14 through the acting section 24 and thus illumination of a skin area to be treated is possible.

In the illustrated embodiment, the recess 32 is open in an area within the acting section in exactly one spatial direction, in direction of the bottom side 28. In this respect, the design of the recess provides, in particular, that it is closed off from an environment when the heating plate 14 is fixed to the printed circuit board at its bottom side 28.

FIG. 5 schematically shows an embodiment of the heating plate 14 according to the invention, in which the connecting section 26 comprises a metal layer 34. By means of the metal layer 34, the heating plate 14 can be soldered onto the printed circuit board. In particular, a copper layer may be provided. In the embodiment shown in FIG. 5, the metal layer 34 comprises multiple areas 34a, 34b, 34c and 34d which are not electrically connected to one another and which can be soldered onto multiple soldering surfaces of the printed circuit board. By using multiple soldering surfaces, it is possible for the soldering surfaces to be directly electrically and thermally connected to soldering surfaces of a heating element, in particular a heating resistor. In addition, the use of further areas that are no longer electrically connected also makes it possible to connect different components with different voltage levels. This can be particularly advantageous for connecting a temperature sensor or various potentials of the heating element, since this is only thermally coupled, but not electrically. Another area of the metal layer 34 can be connected to a ground conductor.

FIG. 5 further shows that the recess 32 is arranged in a first area within the connecting section 26, said first area having a connection to an environment when the heating plate 14 is soldered onto the printed circuit board. In a second area of the recess 32 within the acting section 24, the recess 32 is merely open in one spatial direction (toward the printed circuit board). In this respect, it is possible that the recess 32 has different cross-sections in different areas.

FIG. 6 shows a further embodiment in which the connecting section 26 comprises a metal layer 34. Also shown in FIG. 6 is that the metal layer 34 comprises multiple areas 34a, 34b, 34c, 34d, 34e that are not electrically connected to one another. Different arrangements are possible here. It is understood that further embodiments are also conceivable and advantageous in order to enable different forms of connection in this respect.

FIG. 6 also shows that the recess 32, at least partially, has a bone-shaped cross-section or bone-shaped cross-sectional area in a plane parallel to the heating plate or parallel to the printed circuit board. The bone-shaped cross-sectional area has a central area 36 and two external areas 38a and 38b which are wider than the central area 36. The cross-sectional area is to be understood as an area parallel to the heating plate or parallel to the printed circuit board in the area where the heating plate is attached to the printed circuit board.

FIG. 7 schematically shows an embodiment of the heating plate 14 according to the invention in which the recess 32 is located only within the connecting section 26 and does not extend into the acting section 24. In this respect, the connecting section 26 is comparatively thick. In particular, the connecting section 26 can be designed as a thicker metal layer 34, in particular a copper layer.

The acting section 24 does not comprise any part of the recess 32. Due to the comparatively thick metal layer 34 or the comparatively thick connecting section 26 it is possible for a component to be arranged within the recess 32. The recess 32 is large enough, so to speak, to accommodate a component on the printed circuit board when the heating plate 14 is attached to the printed circuit board.

FIG. 8 schematically shows a perspective view of the bottom side 28 of the heating plate 14 in which the connecting section 26 comprises a metal layer 34. The recess 32 extends only into the connecting section 26, but not into the acting section 24. Due to the fact that the recess 32 has only a comparatively small depth, under certain circumstances no component can be accommodated in the recess 32 if the heating plate 14 is soldered onto a printed circuit board. However, light may nevertheless be transmitted through the printed circuit board and through the acting section 24, for example, by an LED disposed on another side of a printed circuit board (i.e., a side opposite the side of the heating plate), so that the skin area to be treated can be illuminated. This is advantageous, for example, if the material of the acting section 24 is translucent, while the material of the connecting section 26 is not.

FIG. 9 shows a schematic view of an embodiment of the heating plate 14 according to the invention in the area of the bottom side 28. At least in a partial area within the acting section 24, the recess 32 has a bone-shaped cross-section or bone-shaped cross-sectional area. Furthermore, in the illustrated embodiment in FIG. 9, different areas of the metal layer 34 of the connecting section 26 that are not electrically connected to one another are provided, which can be soldered onto multiple soldering surfaces of a printed circuit board.

FIG. 10 shows a schematic view of an embodiment in which the connecting section 26 comprises a comparatively thick metal layer 34 which, in the illustrated embodiment, is also composed of multiple areas 34a, 34b, 34c, 34d, 34e, 34f, 34g. In the illustrated embodiment in FIG. 10, the recess 32 does not extend into the acting section 24, but only into the connecting section 26. Also in FIG. 10, the recess 32 has a substantially bone-shaped cross-sectional area, but only in a partial area within the connecting section 26.

FIG. 11 shows a schematic view of an embodiment of a heating plate 14 according to the invention in which a metal layer 34 of the connecting section 26 comprises multiple areas 34a, 34b, 34c, 34d, 34e, 34f. In addition, the recess 32 is intended to include areas having different depths. In a first area 38a, the recess 32 extends deeper into the acting section 24 than in a second area 38b divided in two parts.

FIG. 12 shows a schematic view of an embodiment of the heating plate 14 according to the invention, in which the recess 32 comprises areas with different depths. In addition, the connecting section 26 comprises a metal layer 34 with different areas.

FIG. 13 shows a schematic view of an embodiment of a heating plate 14 according to the invention, in which the recess 32 is designed as consisting of two parts. In a first area 32a of the recess, for example, a first component can be accommodated, and in a second area 32b of the recess, for example, a second component can be accommodated when the heating plate 14 is attached to the printed circuit board. It is understood that other arrangements and embodiments are also conceivable.

FIG. 14 shows a schematic view of an embodiment in which the heating plate 14 has a further recess 40 on its top side 20 in the acting section 24. In the illustrated embodiment, the recess 40 is designed as consisting of four parts with four areas 40a to 40d. In particular, it is advantageous if the further recess 40 has a depth selected such that a bottom of the further recess 40 can be brought into contact with the skin by pressing in the skin when the acting section 24 is applied to the skin. A depth between 0.03 mm and 0.1 mm has proven to be particularly advantageous for this purpose. In the embodiment shown, the logo lettering is thus only so far offset or so deeply inserted into the top side 20 of the heating plate 14 that contacting the skin is nevertheless possible even in the area of the further recess 40 or the lettering, in order to heat the skin area to be treated.

FIG. 15 schematically shows a design of the components of the device 10 according to the invention. An LED 52 is attached so that light can be directed through the heating plate 14 onto the skin area to be treated. In this regard, it is sufficient that the recess is located in the connecting section, i.e., that an area of the connecting section is provided where no metal surface is applied. Since the ceramic material is translucent, the skin area to be treated can be illuminated.

In this respect, FIG. 16 shows a perspective view, from which it becomes apparent that the printed circuit board 12 can also have a cutout, so that the recess 32 in the heating plate 14 is accessible from below in order to allow light from the LED 52 to pass through.

In FIG. 17, a view of a further advantageous design according to the invention is shown. The illustration consists of a sectional view transverse to a longitudinal axis of the device 10, in which the upper layer of the heating plate 14 or its acting section is not visible (sectional plane striped). A total of 4 heating elements 16a, 16b, 16c, 16d and an LED 52 are arranged in the recess 32.

The invention has been comprehensively described and explained with reference to the drawings and the description. The description and explanation are to be understood as an example and not restrictive. The invention is not limited to the disclosed embodiments. Other embodiments or variations will be apparent to those skilled in the art upon use of the present invention and upon close analysis of the drawings, the disclosure and the following claims.

In the claims, the word “comprising” does not exclude other elements or steps, and the indefinite article “a” or “an” does not exclude a plurality. A single element or single unit may fulfill the functions of several items recited in the claims. An element, a unit, a device and a system may partially or completely be implemented by corresponding hardware and/or software. The mere fact that certain measures are recited in mutually different dependent claims does not indicate that a combination of these measures cannot be used to advantage. Reference signs in the claims are not to be understood restrictively. Identical reference signs in the figures denote the same elements in each case.

1. A heating plate (14) for the thermal-medical treatment of skin, comprising:
an acting section (24) for being applied to the skin on a top side (20) of the heating plate;
a connecting section (26) for attaching the heating plate to a printed circuit board (12) on a bottom side (28) of the heating plate; and
a recess (32) on the bottom side of the heating plate.

2. The heating plate (14) according to claim 1, wherein the recess (32) is designed for accommodating of a component on the printed circuit board (12) when the heating plate is attached to the printed circuit board, in particular an LED (52), preferably a soldered-on surface-mounted LED.

3. The heating plate (14) according to claim 1, wherein the recess (32) comprises areas (32a, 32b) with different depths.

4. The heating plate (14) according to claim 1, wherein the recess (32) extends through the connecting section (26) and into the acting section (24).

5. The heating plate (14) according to claim 1, wherein the recess (32)
in an area within the acting section (24) is open in exactly one spatial direction;
is designed to be closed off from an environment by the printed circuit board (12); and/or
has, at least in a partial area, a bone-shaped cross-sectional area with a central area (36) and two external areas (38a, 38b) widened relative to the central area.

6. The heating plate (14) according to claim 1, wherein
the acting section (24) is formed of a ceramic material; and
the connecting section (26) is preferably formed of a different material connected to the ceramic material using a baking process.

7. The heating plate (14) according to claim 1, wherein
the acting section (24) in the area of the recess (32) has a thickness of between 0.2 mm and 0.8 mm, preferably about 0.4 mm;
the heating plate has a maximum thickness between 0.5 mm and 1.5 mm; and/or
the acting section has rounded edges.

8. The heating plate (14) according to claim 1, comprising
a further recess (40) on the top side (20) of the heating plate in the acting section (24), wherein
the further recess (40, 40a, 40b, 40c, 40d) preferably has a depth which is selected such that a bottom of the further recess is brought into contact with the skin when the acting section is applied to the skin by pressing in the skin.

9. The heating plate (14) according to claim 1, wherein
the connecting section (26) comprises a metal layer (34) for soldering the heating plate onto the printed circuit board (12), in particular a copper layer; and
the metal layer preferably comprises multiple areas (34a, 34b, 34c, 34d, 34e, 34f, 34g) not electrically connected to one another for soldering onto multiple soldering surfaces of the printed circuit board.

10. The heating plate (14) according to claim 1, wherein
an edge section (30) which is set back relative to the acting section (24) on the top side (20) of the heating plate, wherein
the edge section preferably circulates the acting section step-like and/or is formed of the same material as the acting section.

11. A device (10) for thermal-medical treatment of skin, comprising:
a printed circuit board (12), in particular a flexible printed circuit board;
a heating plate (14) according to claim 1, which is attached to the printed circuit board; and
a heating element (16) thermally connected to the heating plate for heating the heating plate to a treatment temperature.

12. The device (10) according to claim 11, comprising a component attached to the printed circuit board (12) in the recess (32) of the heating plate (14), in particular an LED.

13. A method of manufacturing a heating plate (14) according to claim 1, comprising a step of creating the recess (32), a further recess (40, 40a, 40b, 40c, 40d) and/or an edge section (30) by ablative laser processing of the heating plate.

14. A method of manufacturing a heating plate (14), according to claim 1, comprising a step of creating the recess (32), a further recess (40, 40a, 40b, 40c, 40d) and/or an edge section (30) by pressing a ceramic powder into a mold.

15. A The method of manufacturing a heating plate (14) according to claim 13, wherein parallel processing of multiple heating plates in a panel is performed.
